(19) United States
(12) Patent Application Publication
(10) Pub. No.: US 2025/0064311 A1
(43) Pub. Date: Feb. 27, 2025

(54) MEDICAL ASSEMBLIES, DEVICES, SYSTEMS, AND RELATED METHODS FOR DISSIPATING HEAT

(71) Applicant: Boston Scientific Scimed, Inc., Maple Grove, MN (US)

(72) Inventor: Konstantin KOPOTIYENKO, Dedham, MA (US)

(73) Assignee: Boston Scientific Scimed, Inc., Maple Grove, MN (US)

(21) Appl. No.: 18/809,648

(22) Filed: Aug. 20, 2024

Related U.S. Application Data
(60) Provisional application No. 63/578,390, filed on Aug. 24, 2023.

(51) Int. Cl. A61B 1/12; A61B 1/005; A61B 1/008; A61B 1/06

(52) U.S. Cl. CPC A61B 1/128; A61B 1/0055; A61B 1/0057; A61B 1/008; A61B 1/0676; A61B 1/0684

(57) ABSTRACT

A medical device may include a handle, an insertion portion coupled to the handle, and a distal tip coupled to a distal end of the insertion portion. The insertion portion may be configured to be inserted into a body lumen of a subject. The distal tip may include a light emitting diode (LED) and a heat sink. The heat sink may be configured to absorb heat generated by the LED.

### CROSS-REFERENCE TO RELATED APPLICATIONS

This application claims the benefit of priority of U.S. Provisional Patent Application No. 63/578,390, filed on Aug. 24, 2023, the entirety of which is incorporated herein by reference.

### TECHNICAL FIELD

This disclosure generally relates to medical assemblies, devices, systems, and related methods for dissipating heat. More particularly, at least some embodiments of this disclosure relate to assemblies, devices, systems, and related methods for managing the temperature of a distal tip of a medical device.

### BACKGROUND

Medical devices are often inserted into the body to perform a therapeutic and/or diagnostic procedure inside a subject's body. An example of such a device is an endoscope or other type of scope or insertion device, which includes an insertion portion that is introduced into the body for diagnostic or therapeutic purposes. An insertion portion of an endoscope is inserted into the subject's body through an opening (e.g., a natural opening or an incision) and is delivered to a site inside the body, for example, through a body lumen. In one example, an endoscope may be inserted into a subject's mouth and advanced through the subject's esophagus.

Endoscopes or other scopes may include a variety of features, for example, to assist in performing a therapeutic and/or diagnostic procedure inside the subject's body. For example, a distal tip of an endoscope may include an imaging device and one or more illumination devices, such as, for example, one or more light emitting diodes (LEDs). The LEDs illuminate the interior of the subject's body (e.g., body lumen), thus allowing a user to visualize the interior of the subject's body. The distal tip may also include one or more openings (e.g., of a working channel), elevators, or other features. During use, the one or more components of the distal tip may generate heat. For example, the one or more LEDs may generate heat, which may be harmful to the subject and/or affect the performance of the LEDs, imaging devices, and/or other components of the distal tip. Size constraints of the distal tip may limit the number or types of components that may be included, for example, to manage the heat generated by the LEDs or other components of the distal tip. Therefore, medical assemblies, devices, systems, and related methods are needed for incorporating heat management components in a distal tip of an endoscope. The assemblies, devices, systems, and methods of this disclosure may rectify some of the deficiencies described above or address other aspects of the art.

### SUMMARY

Examples of this disclosure relate to, among other things, assemblies, devices, systems, and methods for performing one or more medical procedures with medical devices. Specifically, this disclosure includes medical assemblies, devices, and systems comprising at least one LED, along with methods of use thereof (e.g., methods of managing the heat generated by one or more components of the distal tip). Each of the examples disclosed herein may include one or more of the features described in connection with any of the other disclosed examples.

According to one aspect, a medical device may comprise a handle, an insertion portion coupled to the handle, and a distal tip coupled to a distal end of the insertion portion. The insertion portion may be configured to be inserted into a body lumen of a subject. The distal tip may include a light emitting diode (LED) and a heat sink. The heat sink may be configured to absorb heat generated by the LED.

The medical device may include one or more of the following features. In some aspects, the heat sink may be disposed proximally of the LED in the distal tip. The heat may include a ferrule and an articulation wire. The heat sink may be coupled to a distal end of the articulation wire. Each of the ferule and the articulation wire may be comprised of one or more thermally conductive materials.

In some aspects, a space may be defined between the LED and the heat sink. The space may include at least one of a thermally conductive material, a thermally conductive epoxy, or a thermally conductive adhesive. The at least one of the thermally conductive material, the thermally conductive epoxy, or the thermally conductive adhesive of the space may be configured to contact both the LED and the heat sink.

In some aspects, the LED may be a first LED, and the heat sink may be a first heat sink. The distal tip may further include a second LED and a second heat sink. The second heat sink may be disposed proximally of the second LED. A first space may be defined between the first LED and the first heat sink. A second space may be defined between the second LED and the second heat sink.

In some aspects, the first space may include at least one of a first thermally conductive material, a first thermally conductive adhesive, or a first thermally conductive epoxy. The second space may include at least one of a second thermally conductive material, a second thermally conductive epoxy, or a second thermally conductive adhesive. In some aspects, at least one of the first thermally conductive material, the first thermally conductive epoxy, or the first thermally conductive adhesive of the first space may contact both the first LED and the first heat sink. The at least one of the second thermally conductive material, the second thermally conductive epoxy, or the second thermally conductive adhesive of the second space may contact both the second LED and the second heat sink.

In some aspects, the heat sink may include an articulation joint. The articulation joint may be configured to articulate the distal tip. The articulation joint may include one or more gaps. The articulation joint may be disposed in a distal portion of the insertion portion. A space may be defined between the LED and the articulation joint. The space may include at least one of a thermally conductive material, a thermally conductive adhesive, or a thermally conductive epoxy.

In some aspects, the heat sink may include at least one layer of the insertion portion. The at least one layer may include a thermally conductive mesh. A space may be defined between the LED and the at least one layer of the insertion portion. The space may include at least one of a thermally conductive material, a thermally conductive adhesive, or a thermally conductive epoxy.

According to another aspect, a distal end portion of a medical device may include a first LED, a second LED, and a heat sink. The heat sink may be disposed proximally of the first LED and the second LED. The heat sink may be configured to absorb heat from the first LED and the second LED.

The medical device may include one or more of the following features. In some examples, a first space may be defined between the first LED and the heat sink. A second space may be defined between the second LED and the heat sink. Each of the first space and the second space may include a thermally conductive adhesive.

In some examples, the thermally conductive adhesive of the first space may contact both the first LED and the heat sink. The thermally conductive adhesive of the second space may contact both the first LED and the heat sink. The thermally conductive adhesive of the second space may contact both the second LED and the heat sink.

According to another aspect, a distal end portion of a medical device may comprise a first LED, a second LED, a first heat sink, and a second heat sink. The first heat sink may be disposed proximally of the first LED. The second heat sink may be disposed proximally of the second LED. Each of the first heat sink and the second heat sink may be configured to absorb heat form the first LED and the second LED, respectively.

The medical device may include one or more of the following features. In some aspects, a first space may be defined between the first LED and the first heat sink. A second space may be defined between the second LED and the second heat sink. Each of the first space and the second space may include at least one of a thermally conductive adhesive, a thermally conductive epoxy, or a thermally conductive material.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a medical system, according to aspects of this disclosure.

FIG. 2 illustrates a schematic, cross-sectional view of a distal end of a medical device of the medical system of FIG. 1, according to aspects of this disclosure.

FIG. 3 illustrates a schematic, cross-sectional view of an alternative configuration of a distal tip of the medical device of the medical system of FIG. 1, according to aspects of this disclosure.

FIG. 4 illustrates a schematic, cross-sectional view of another alternative configuration of a distal tip of the medical device of the medical system of FIG. 1, according to aspects of this disclosure.

FIG. 5 illustrates a schematic, cross-sectional view of yet another alternative configuration of a distal tip of the medical device of the medical system of FIG. 1, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, terms that indicate the geometric shape of a component/surface encompass both exact and approximate shapes.

Although a target site is discussed herein as being in a subject's gastrointestinal tract, this disclosure is not so limited, as the target site may be any internal lumen, organ, cavity, or other tissue within the subject. Additionally, although endoscopes are referenced herein, it will be appreciated that the disclosure encompasses various devices that may be inserted into a lumen of a subject, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

A distal tip of an endoscope may include many components, including, for example, one or more imaging devices (e.g., cameras), lighting elements (e.g., light emitting diodes, or LEDs), openings, etc. The LEDs may be used to provide illumination within a subject's body. For example, the LEDs may be used (e.g., in combination with the imaging device) to enable a user to visualize and navigate inside the subject's body without the need for invasive surgery. During use, one or more components of the distal tip may generate heat. For example, the LEDs may generate heat. The heat generated by the LEDs of the distal tip may be harmful to the subject, reduce efficiency of the LEDs, and/or damage the LEDs. Embodiments of the disclosure may address one or more of the limitations in the art. The scope of the disclosure, however, is defined by the attached claims and not the ability to solve a specific problem.

The disclosure is drawn to assemblies, devices, systems, and related methods, for dissipating the heat generated by one or more components (e.g., LEDs) of a distal tip of an endoscope, among other aspects. In some aspects, a distal tip of the endoscope may include a one or more heat sinks. The one or more heat sinks may be configured to dissipate heat generated by the one or more LEDs. In other aspects, the one or more heat sinks may be configured to dissipate heat generated by other components of the distal tip of the medical device.

This disclosure is described with reference to exemplary medical devices, assemblies, and/or systems for dissipating heat generated by the one or more components of the distal tip of the endoscope (e.g., LEDs). This may provide improved medical tool functionality and/or assist medical professionals with performing medical procedures. However, it should be noted that reference to any particular device and/or any particular procedure is provided only for convenience and not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. This disclosure may be understood with reference to the following description and the appended drawings, wherein like elements are referred to with the same reference numerals.

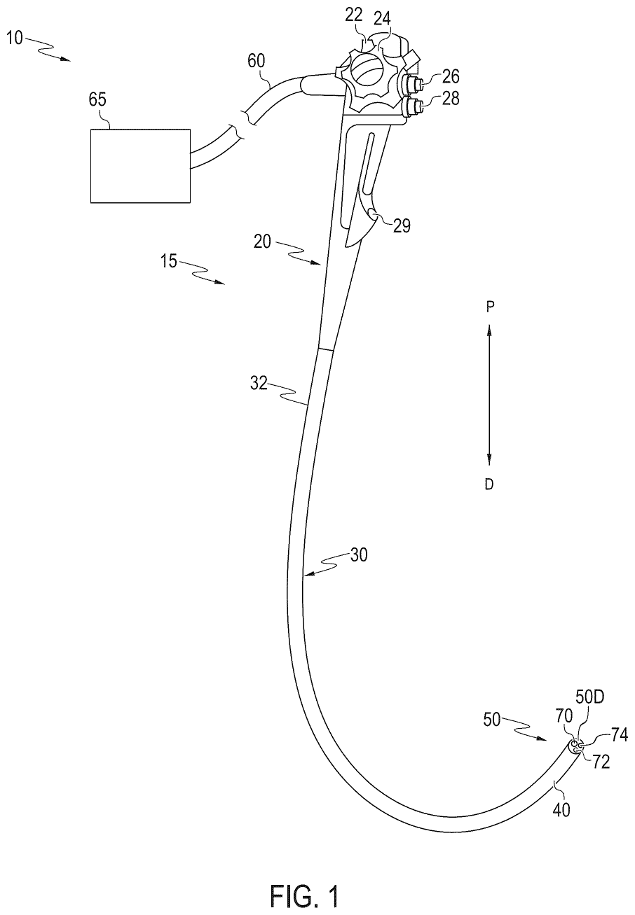

Referring to FIG. 1, a medical system 10 according to an exemplary embodiment is shown. Medical system 10 may include a medical device 15 comprising a handle 20 and an insertion portion 30 (e.g., a shaft or a catheter). Insertion portion 30 may be connected to and extend from a distal portion of handle 20. Insertion portion 30 may terminate distally in a distal tip 50. An umbilicus 60 may extend from a proximal portion of handle 20. As discussed in detail below, a distal face 50D of distal tip 50 may include one or more distal openings 70 (e.g., distal openings of working channels or lumens). Moreover, in some aspects, distal face 50D includes one or more imaging devices 72 and/or one or more illumination devices (e.g., LEDs) 74. As discussed in detail below, distal tip 50 includes one or more components to help dissipate or otherwise direct heat, for example, heat generated by illumination device(s) (e.g., LEDs) 74, and/or for suction, irrigation, insufflation, accessory devices, etc. In some examples, distal tip 50 may further include one or more end effectors (e.g., graspers, electrodes, biopsy devices, etc.).

Distal end portion 40 may further include one or more imaging devices 72. Imaging device(s) 72 may include one or more cameras, image sensors, lenses, etc. Distal end portion 40 may also include one or more illumination devices, such as, for example, one or more LEDs 74. In some examples, imaging device(s) 72 and/or LEDs 74 may be disposed on distal face 50D of distal tip 50. Additionally or alternatively, imaging device(s) 72 and/or LEDs 74 may be disposed on a side surface of distal end portion 40, for example, at or adjacent to distal tip 50. In further examples, imaging device(s) 72 and/or LEDs 74 may be disposed anywhere along a length of insertion portion 30 and/or distal end portion 40.

Umbilicus 60 may be removably coupled (e.g., directly or indirectly) to a processing unit 65. Processing unit 65 may be configured to process information (e.g., sensor data, imaging data, light data, etc.) received from medical device 15. In some aspects, processing unit 65 may be a controller associated with medical device 15. Imaging device(s) 72 and/or LEDs 74 may be electrically coupled (e.g., directly or indirectly) to processing unit 65, for example, via one or more wires and/or cables extending through insertion portion 30, through handle 20, and through umbilicus 60. For example, umbilicus 60 may include one or more electrical cables and/or light cables for coupling to processing unit 65 via, e.g., a removable connector.

Although not shown, processing unit 65 may include a visual output (e.g., an embedded monitor or screen), or processing unit 65 may be coupled to a visual output (e.g., an external or separate monitor or screen coupled to processing unit 65). Although not shown, umbilicus 60 may additionally or alternatively include one or more lumens for supplying a gas or a liquid, or applying suction or negative pressure, to handle 20 and/or insertion portion 30. For example, the gas or the liquid may be applied, or delivered, through the working channel of medical device 15.

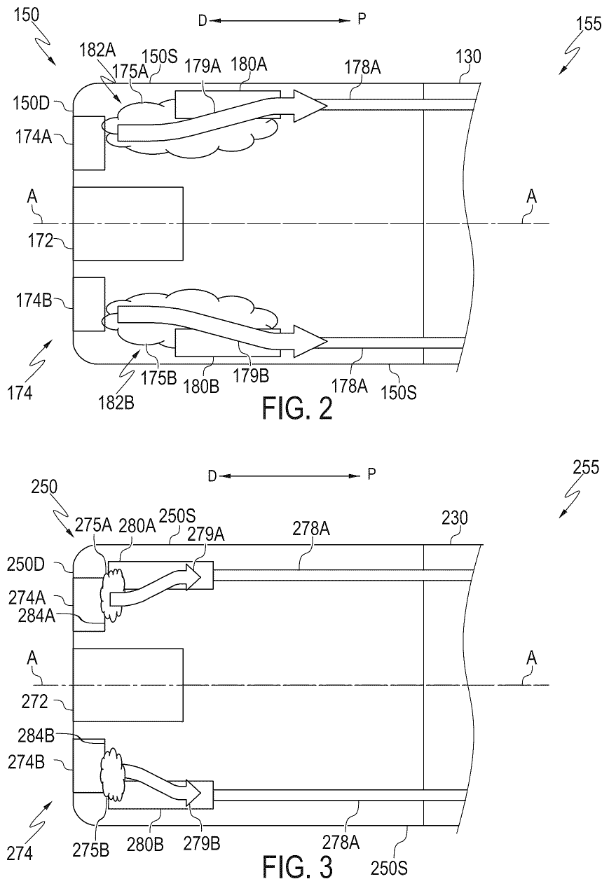

Handle 20 may include one or more actuators, for example, a first actuator 22 and/or a second actuator 24. First actuator 22 and/or second actuator 24 may include, for example, rotatable knobs that rotate to push/pull one or more elements (e.g., steering/articulation wires or cables) that extend through one or more portions of insertion portion 30 and connect to a distal end portion 40 of insertion portion 30. For example, first actuator 22 and/or second actuator 24 may be configured to rotate about a respective axis to push/pull actuating elements (e.g., wires or cables shown in FIGS. 2 and 3) that extend within one or more lumens of insertion portion 30. Rotation of first actuator 22 and/or second actuator 24 may cause a portion of insertion portion 30 (e.g., distal end portion 40) to bend, for example, via an articulating joint (not shown). Additionally or alternatively, handle 20 may include one or more additional actuators (e.g., buttons, knobs, levers, locks, etc.) to, for example, limit movement of the first actuator 22 and/or second actuator 24, close or open an end effector, rotate an end effector about a longitudinal axis, raise or lower an elevator to move a device delivered through the working channel, capture an image and/or video, and/or provide other functionality to an end effector and/or distal tip 50.

Handle 20 may also include one or more valves, for example, a first valve 26 and a second valve 28. Although two valves (i.e., first valve 26 and second valve 28) are shown, handle 20 may include additional (e.g., a third valve, a fourth valve, etc.) or fewer valves (e.g., no valves or only first valve 26). In some embodiments, first valve 26 may be configured to control the supply of air and/or water to distal tip 50. Second valve 28 may be configured to control the application of suction to distal tip 50. Additional valves may be used, for example, to control the application of one or more medicines, agents, materials, etc. from distal tip 50.

Handle 20 may also include a port or proximal opening 29, which may be fluidly connected to one or more lumens or working channel of insertion portion 30. For example, a medical instrument (not shown) may be inserted into proximal opening 29 and may be extended to and/or distally from distal tip 50 via the one or more lumens. The lumen(s) may have a distal opening 70 on distal face 50D of distal tip 50, which may be a distalmost end of distal end portion 40. Distal opening 70 may be fluidly coupled to proximal opening 29 of handle 20. In such a way, a medical instrument inserted into proximal opening 29 may be extended distally to distal opening 85. In some aspects, the medical instrument may be extended distally from distal opening 85, for example, distal of distal tip 50. Additionally or alternatively, one or more materials (e.g., liquids, gels, gasses, patches, powders, etc.) may be supplied to a target site via distal opening 85. Additionally or alternatively, suction may be applied to the target site from distal opening 70 (e.g., via proximal opening 29), for example, to remove fluid and/or debris from the target site.

One or more portions of insertion portion 30 may be flexible and may be formed of any medical grade material suitable for accessing a tortuous path within the body. The rigidity/flexibility of insertion portion 30 is not limited. In some embodiments, the rigidity/flexibility of insertion portion 30 may vary, for example, along a longitudinal length of insertion portion 30. A longitudinal length of insertion portion 30 may vary and is not limited. For example, insertion portion 30 may have a length of approximately 5-100 inches, for example, between 10-70 inches. Furthermore, an outer diameter of insertion portion 30 is not limited. For example, the outer diameter of insertion portion 30 may be approximately 0.07-0.60 inches, for example, between 0.10-0.50 inches. In some embodiments, the outer diameter of insertion portion 30 may vary along the longitudinal length. For example, the outer diameter of a proximal portion 32 of insertion portion 30 may be less than or greater than the outer diameter of distal end portion 40 of insertion portion 30, or vice versa.

A cross-section of insertion portion 30 and/or distal tip 50 may be generally circular, ovular, or any other shape commonly used in the art for the insertion portion of a medical device. In other examples, the cross-section of insertion portion 30 and/or distal tip 50 may be square, rectangular, hexagonal, polygonal, or any other shape. In some examples, insertion portion 30 may have a different cross-sectional shape than distal tip 50. For example, insertion portion 30 may have a circular cross-sectional shape, and distal tip 50 may have a hexagonal cross-sectional shape, or vice versa. The size and shape of insertion portion 30 and/or distal tip 50 is not limited to the size and/or shapes described herein and may be any other shape commonly used in the art.

As mentioned above, although not shown, distal end portion 40 may include one or more articulation joints configured to articulate distal tip 50 in one or more directions (e.g., left, right, up, down, etc.). Although not shown, distal end portion 40 may further include one or more treatment or accessory devices (e.g., laser fibers, elevators, etc.), and/or one or more other devices to otherwise image, view, or otherwise treat a target site.

FIG. 2 illustrates a schematic, cross-sectional view of a distal portion 155 of an insertion portion 130 of a medical device (e.g., medical device 15). Distal portion 155 includes a distal tip 150. Distal tip 150 may be utilized with medical device 15, shown in FIG. 1. Distal tip 150 may have any or all of the features and/or characteristics of distal tip 50, discussed above with respect to FIG. 1. For example, insertion portion 130 may terminate at a distal face 150D of distal tip 150. As shown, distal portion 155, for example, including insertion portion 130 and distal tip 150, may include a longitudinal axis A.

In some examples, distal tip 150 may include an imaging device 172 and/or one or more illumination devices, for example, LEDs 174. For example, distal tip 150 may include a first LED 174A and a second LED 174B. Imaging device 172 and/or LEDs 174 may each be disposed on distal face 150D of distal tip 150. At least a portion of distal face 150D may be perpendicular to axis A. In some examples, imaging device 172 may be disposed on distal face 150D, for example, between first LED 174A and second LED 174B. Although not shown, alternative configurations of imaging device 172, first LED 174A, and/or second LED 174B may be utilized with distal tip 150. For example, first LED 174A may be disposed between imaging device 172 and second LED 174B. Although not shown, distal face 150D may include one or more openings, for example, one or more working channels and/or lumens in insertion portion 130. Additionally, imaging device 172, first LED 174A, and/or second LED 174B may be positioned on distal face 150D in various positions relative to the one or more working channels and/or lumens.

Imaging device 172, first LED 174A, and second LED 174B may be oriented such that a straight line intersects each of imaging device 172, first LED 174A, and second LED 174B. For example, each of imaging device 172, first LED 174A, and second LED 174B may be oriented on a same plane, for example, the cross-sectional plane shown in FIG. 2. In other examples, one or more of Imaging device 172, first LED 174A, and/or second LED 174B may be oriented on different planes. For example, a straight line may intersect just imaging device 172 and first LED 174A or second LED 174B. Alternatively, imaging device 172, first LED 174A, and second LED 174B may be oriented such that a straight line intersects just first LED 174A and second LED 174B and not imaging device 172. Other configurations of each of imaging device 172, first LED 174A, and second LED 174B may be contemplated.

Although one imaging device 172 and two LEDs (e.g., first LED 174A and second LED 174B) are shown in FIG. 2, distal tip 150 may include additional imaging devices and/or illumination devices. For example, the additional imaging devices and/or illumination devices (e.g., LEDs) disposed on distal face 150D and/or on one or more side surfaces 150S of distal tip 150. In some examples, distal tip 150 may include just one LED (e.g., first LED 174A or second LED 174B). In other examples, distal tip 150 may include additional LEDs (e.g., three LEDs, four LEDs, etc.) and/or additional imaging devices.

In some examples, distal tip 150 may further comprise one or more steering, or articulation, wires or cables. For example distal tip 150 may comprise a first steering cable 178A and a second steering cable 178B. Each of first steering cable 178A and second steering cable 178B may extend proximally from distal tip 150. For example, first steering cable 178A and second steering cable 178B may extend parallel to axis A through a portion of distal tip 150 and through insertion portion 130 to a handle (not shown). Although not shown, a proximal end of each of first steering cable 178A and second steering cable 178B may extend through insertion portion 130 and couple to one or more steering components (not shown) disposed within handle 20 of medical device 15. For example, the one or more steering components may be coupled (e.g., directly or indirectly) to first actuator 22 and/or second actuator 24. As such, movement of first steering cable 178A and/or second steering cable 178B may be controlled by first actuator 22 and/or second actuator 24. For example, rotation of first actuator 22 and/or second actuator 24 in a first direction (e.g., clockwise or counterclockwise) may rotate or otherwise move the one or more steering components in the same first direction. Similarly, rotation of first actuator 22 and/or second actuator 24 in a second direction (e.g., counterclockwise or clockwise) may rotate or otherwise move the one or more steering components in a second direction.

A distal end of each steering cable may include or be coupled to a termination, which may include an anchor, a crimp, and/or a ferrule. For example, a distal end of first steering cable 178A may include or be coupled to a first ferrule 180A, and a distal end of second steering cable 178B may include or be coupled to a second ferrule 180B. Additional steering cables (not shown) may also include or be coupled to a ferrule. For example, a third steering cable may include or be coupled to a third ferrule, a fourth steering cable may include or be coupled to a fourth ferrule, etc. Each ferrule (e.g., first ferrule 180A, second ferrule 180B, etc.) may be fixed to the distal end of each steering cable (e.g., first steering cable 178A, second steering cable 178B, etc.) by, for example, one or more of an adhesive, an epoxy, a crimp, a swage, a solder, a weld, or any mechanisms or techniques commonly used in the art. In some examples, for example, when the steering wires or cables are coupled to the ferrules via one or more adhesives or epoxies, the one or more adhesives or epoxies may include one or more thermally conductive materials (i.e., capable of or configured to conduct heat). In further examples, any combination of coupling mechanisms or techniques may be used. For example, first ferrule 180A may be coupled to first steering cable 178A by an adhesive and a crimp, and second ferrule 180B may be coupled to second steering cable 178B by a swage and an epoxy.

Although not shown, in some examples, distal tip 150 may include one or more features (e.g., through holes, lumens, openings, etc.), for example, to assist in securing, or coupling, each steering cable to distal tip 150 via each respective ferrule (e.g., first ferrule 180A and/or second ferrule 180B). For example, distal tip 150 may include one or more features to secure first ferrule 180A to distal tip 150. Additionally or alternatively, distal tip 150 may include one or more features to secure second ferrule 180B to distal tip 150. Additionally or alternatively, each ferrule may be coupled to distal tip 150 via one or more adhesives and/or epoxies. In some examples, the adhesive(s) and/or epoxy(ies) may include one or more thermally conductive materials.

One or more of each ferrule and/or steering cable may include one or more thermally conductive materials. For example, first steering cable 178A, second steering cable 178B, first ferrule 180A, and/or second ferrule 180B may include (e.g., be comprised of) one or more thermally conductive materials (e.g., silver, copper, steel, aluminum, bronze, gold, etc.). Accordingly, heat generated by first LED 174A may be at least partially absorbed and/or distributed by first steering cable 178A and/or first ferrule 180A. Similarly, heat generated by second LED 174B may be at least partially absorbed and/or distributed by second steering cable 178B and/or second ferrule 180B.

Each of first steering cable 178A, second steering cable 178B, first ferrule 180A, and second ferrule 180B may be contained by, enclosed by, or otherwise at least partially insulated within distal tip 150 and insertion portion 130. In these aspects, respective portions of each of first steering cable 178A, second steering cable 178B, first ferrule 180A, and second ferrule 180B may be insulated within distal tip 150 and insertion portion 130 to protect the subject as first steering cable 178A, second steering cable 178B, first ferrule 180A, and/or second ferrule 180B increase in temperature. For example, the temperature of each of first steering cable 178A, second steering cable 178B, first ferrule 180A, and/or second ferrule 180B may increase as the heat is conducted away from the respective LEDs (e.g., first LED 174A or second LED 174B).

In some examples, distal tip 150 and/or insertion portion 130 may be comprised of one or more materials, for example to assist in protecting the subject from the heat generated by the LEDs and absorbed by first steering cable 178A, second steering cable 178B, first ferrule 180A, and/or second ferrule 180B. For example, distal tip 150 and/or insertion portion 130 may be comprised of one or more insulating materials (e.g., polyvinyl chloride, thermoplastic polyurethane, polyetheretherketone, perfluoroalkoxy, ethylene tetrafluoroethylene, thermoplastic elastomers, ceramic(s), etc.). Additionally or alternatively, distal tip 150 and/or insertion portion 130 may be comprised of one or more layers of insulating materials, for example, surrounding an external surface of distal tip 150 and/or insertion portion 130. In these aspects, distal tip 150 and insertion portion 130 may radially surround each of first steering cable 178A, second steering cable 178B, first ferrule 180A, and second ferrule 180B. Additionally, distal tip 150 and/or insertion portion 130 may help to protect one or more components of medical device 15 (e.g., imaging device 172).

As illustrated in FIG. 2, a first space 182A may be defined between first LED 174A and first ferrule 180A. For example, first LED 174A and first ferrule 180A may be separated via first space 182A such that first LED 174A and first ferrule 180A are not in direct contact with one another. Similarly, a second space 182B may be defined between second LED 174B and second ferrule 180B such that second LED 174B and second ferrule 180B are not in direct contact with one another.

In some examples, at least a portion of each of first space 182A and/or second space 182B may be filled with a thermally conductive material, for example, to assist in absorbing and transferring heat from each LED (e.g., first LED 174A and second LED 174B) to each respective ferrule (e.g., first ferrule 180A and second ferrule 180B). For example, each space (e.g., first space 182A and second space 182B) may be filled with or include a thermally conductive adhesive, a thermally conductive epoxy, a metallic material (e.g., copper, steel, etc.) or any other thermally conductive material. As such, the thermally conductive material within each space (e.g., first space 182A and second space 182B) may be in contact each LED and each ferrule. For example, the thermally conductive material within first space 182A may contact first LED 174A and first ferrule 180A. Similarly, the thermally conductive material within second space 182B may contact second LED 174B and second ferrule 180B. In some examples, first space 182A and second space 182B may be filled with the same thermally conductive material. In other examples, first space 182A and second space 182B may each be filled with different thermally conductive materials.

The presence of the one or more thermally conductive materials is illustrated schematically in FIG. 2 by clouds, for example, a first cloud 175A and a second cloud 175B. Although first cloud 175A and second cloud 175B are illustrated as being at least partially between each respective LED and ferrule, the thermally conductive material(s) may be elsewhere within distal tip 150. For example, the thermally conductive material(s) may be proximal to imaging device 172, and/or may the thermally conductive material(s) may be disposed across an entire cross-sectional width of distal tip 150 (e.g., between side surfaces 150S).

The heat transferred from first LED 174A and second LED 174B is illustrated by a first arrow 179A and a second arrow 179B. For example, at least a portion of the heat generated by each LED (e.g., first LED 174A and second LED 174B) may be transferred from each LED, through the thermally conductive material(s) of each space (e.g., first cloud 175A of first space 182A and second cloud 175B of second space 182B). The heat may then be absorbed or otherwise dissipated by each respective ferrule (e.g., first ferrule 180A and second ferrule 180B) and/or each respective steering cable (e.g., first steering cable 178A and second steering cable 178B).

FIG. 3 illustrates an alternative configuration of a distal portion 255 of an insertion portion 230 of a medical device (e.g., medical device 15). Distal portion 255 includes a distal tip 250. Distal tip 250 may have any or all of the features and/or characteristics of distal tip 150, except as described below. For example, distal tip 250 may include an imaging device 272 and/or one or more LEDs 274. For example, distal tip 250 may include a first LED 274A and a second LED 274B. Imaging device 272 and/or LEDs 274 may each be disposed on a distal face 250D of distal tip 250. Distal face 250D may be perpendicular to longitudinal axis A. In some examples, imaging device 272 may be disposed on distal face 250D, between first LED 274A and second LED 274B. Although not shown, alternative configurations of imaging device 272, first LED 274A, and/or second LED 274B may be utilized with distal tip 250. For example, first LED 274A may be disposed between imaging device 272 and second LED 274B. Additionally or alternatively, imaging device 272, first LED 274A, and/or second LED 274B may be disposed on one or more side surfaces 250S of distal tip 250.

Although not shown, distal face 250D may include one or more openings, for example, one or more working channels and/or lumens in insertion portion 230. Additionally, imaging device 272, first LED 274A, and/or second LED 274B may be positioned on distal face 250D in various positions relative to the one or more working channels and/or lumens.

Distal tip 250 may be disposed on a distalmost end of an insertion portion 230. Insertion portion 230 may have any or all of the features or characteristics of insertion portion 30 described with respect to FIG. 1 and/or insertion portion 130 described with respect to FIG. 2.

Additionally, distal tip 250 may include one or more ferrules and one or more steering cables. For example, distal tip 250 may include a first ferrule 280A coupled to a distal end of a first steering cable 278A, and/or a second ferrule 280B coupled to a distal end of a second steering cable 278B. Each ferrule (e.g., first ferrule 280A and/or second ferrule 280B) and/or each steering cable (e.g., first steering cable 278A and/or second steering cable 278B) may have any or all of the features or characteristics of the ferrules and/or steering cables discussed above with respect to FIG. 2.

In this configuration, each ferrule may contact, or abut, a distal surface of a respective LED. For example, a distal portion of first ferrule 280A may contact, or abut, a proximal surface 284A of first LED 274A. Similarly, a distal portion of second ferrule 280B may contact, or abut, a proximal surface 284B of second LED 274B. In some examples, each ferrule may be coupled to (e.g., directly or indirectly) the respective LED.

Distal tip 250 may further comprise one or more thermally conductive materials, the presence of which is/are illustrated schematically in FIG. 3 by clouds, for example, a first cloud 275A and a second cloud 275B. The one or more thermally conductive materials may include any or all of the thermally conductive materials described above with respect to FIG. 2.

In these aspects, heat generated by the LEDs may be transferred to, and/or absorbed by, each respective ferrule. The heat generated by each respective LED is transferred through the thermally conductive material(s) (e.g., through first cloud 275A and/or second cloud 275B) to each respective ferrule. The heat transferred by each respective LED is illustrated schematically in FIG. 3 by arrows, for example, a first arrow 279A and a second arrow 279B. For example, with first ferrule 280A contacting, abutting, or being coupled to first LED 274A (e.g., via a proximal portion or a proximal end of first LED 274A), heat generated by first LED 274A may be transferred through the thermally conductive material(s) (e.g., through first cloud 275A) and absorbed or otherwise dissipated by first ferrule 280A and/or first steering cable 278A. Similarly, with second ferrule 280B contacting, abutting, or being coupled to second LED 274B (e.g., via a proximal portion or a proximal end of second LED 274B), heat generated by second LED 274B may be transferred through the thermally conductive material(s) (e.g., through second cloud 275B) absorbed by second ferrule 280B and/or second steering cable 278B.

In such a way, the heat may be absorbed or otherwise dissipated by each respective ferrule (e.g., first ferrule 280A and second ferrule 280B) and/or each respective steering cable (e.g., first steering cable 278A and second steering cable 278B). Accordingly, the temperature of each LED (e.g., first LED 274A and/or second LED 274B) and/or the area around each LED (e.g., distal tip 250) may be reduced during the use of the one or more of the LEDs.

As previously discussed, each of first steering cable 278A, second steering cable 278B, first ferrule 280A, and second ferrule 280B may be contained by, enclosed by, or otherwise at least partially insulated within distal tip 250 and insertion portion 230. In these aspects, respective portions of each of first steering cable 278A, second steering cable 278B, first ferrule 280A, and second ferrule 280B may be insulated within distal tip 250 and insertion portion 230 to protect the subject and/or other components of distal tip 250 as first steering cable 278A, second steering cable 278B, first ferrule 280A, and/or second ferrule 280B increase in temperature. For example, the temperature of each of first steering cable 278A, second steering cable 278B, first ferrule 280A, and/or second ferrule 280B may increase as the heat is conducted away from the respective LEDs (e.g., first LED 274A or second LED 274B).

In some examples, distal tip 250 and/or insertion portion 230 may be comprised of one or more materials, for example to assist in protecting the subject from the heat generated by the LEDs and absorbed or otherwise dissipated by first steering cable 278A, second steering cable 278B, first ferrule 280A, and/or second ferrule 280B. For example, distal tip 250 and/or insertion portion 230 may be comprised of one or more insulating materials (e.g., polyvinyl chloride, thermoplastic polyurethane, polyetheretherketone, perfluoroalkoxy, ethylene tetrafluoroethylene, thermoplastic elastomers, ceramic(s), etc.). Additionally or alternatively, distal tip 250 and/or insertion portion 230 may be comprised of one or more layers of insulating materials, for example, surrounding an external surface of distal tip 250 and/or insertion portion 230. In these aspects, distal tip 250 and insertion portion 230 may radially surround each of first steering cable 278A, second steering cable 278B, first ferrule 280A, and second ferrule 280B. Additionally, distal tip 250 and/or insertion portion 230 may help to protect one or more components of medical device 15 (e.g., imaging device 272).

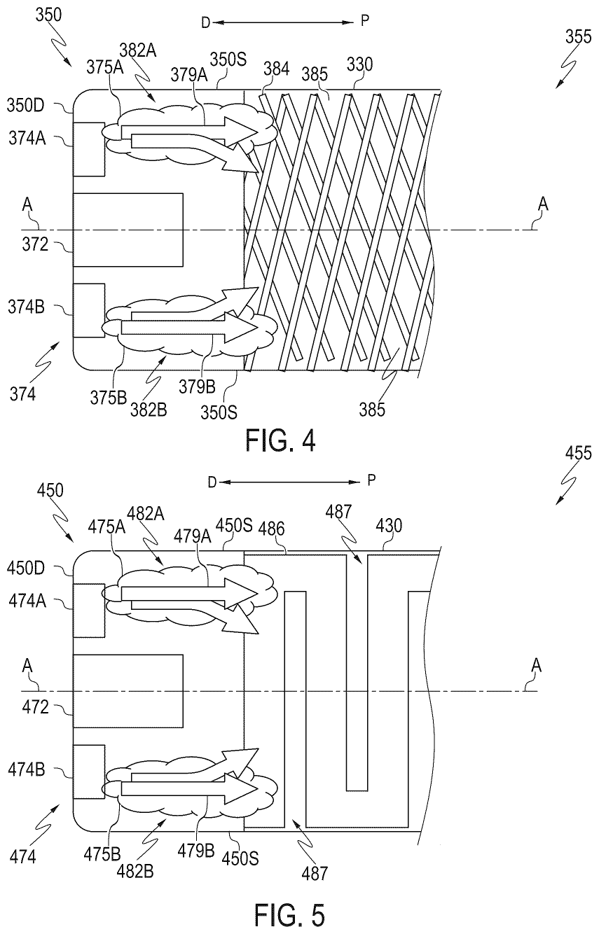

FIG. 4 illustrates an alternative configuration of a portion 355 comprising distal tip 350 and a distal portion of insertion portion 330. Distal tip 350 may have any or all of the features or characteristics of distal tip 150 and/or distal tip 250, except as described below. For example, distal tip 350 may include an imaging device 372 and/or one or more LEDs 374. For example, distal tip 350 may include a first LED 374A and a second LED 374B. Imaging device 372 and/or LEDs 374 may each be disposed on a distal face 350D of distal tip 350. Distal face 350D may be perpendicular to longitudinal axis A. In some examples, imaging device 372 may be disposed on distal face 350D, between first LED 374A and second LED 374B. Although not shown, alternative configurations of imaging device 372, first LED 374A, and/or second LED 374B may be utilized with distal tip 350. For example, first LED 374A may be disposed between imaging device 372 and second LED 374B. Additionally or alternatively, imaging device 372, first LED 374A, and/or second LED 374B may be disposed on one or more side surfaces 350S of distal tip 350.

Although not shown, distal face 350D may include one or more openings, for example, one or more working channels and/or lumens in insertion portion 330. Additionally, imaging device 372, first LED 374A, and/or second LED 374B may be positioned on distal face 350D in various positions relative to the one or more working channels and/or lumens.

Distal tip 350 may be disposed on a distalmost end of insertion portion 330. Insertion portion 330 may have any or all of the characteristics of insertion portion 30, 130, 230 described with respect to FIGS. 1, 2, and 3, respectively. In some examples, insertion portion 330 may be comprised of one or more layers. The one or more layers may be configured to provide structural integrity to insertion portion 330. As such, the one or more layers may circumferentially surround insertion portion 330. For example, the one or more layers may be configured to help prevent kinks or twists of insertion portion 330 while permitting insertion portion 330 to conform, or bend, in the internal curvatures of the subject.

In some examples, insertion portion 330 may include one or more layers 384 comprised of a mesh, a webbing, a threading, a braid, or a lattice. Layer 384 may extend an entirety of insertion portion 330 (e.g., from a distal end to a proximal end), or may extend in discrete lengths of insertion portion 330. Layer 384 may abut or contact distal tip 350. Layer 384 may be comprised of or include any thermally conductive material, such as, for example, stainless steel, copper, aluminum, gold, tungsten, or any other material with thermally conductive properties.

As illustrated in FIG. 4, a first space 382A may be defined between first LED 374A and layer 384. For example, first LED 374A and layer 384 may be separated via first space 382A, such that first LED 374A and layer 384 are not contacting one another. Similarly, a second space 382B may be defined between second LED 374B and layer 384, such that second LED 374B and layer 384 are not contacting one another.

In some examples, each of first space 382A and/or second space 382B may be filled with a thermally conductive material, for example, to assist in absorbing and transferring heat from each LED (e.g., first LED 374A and second LED 374B) to layer 384. For example, each space (e.g., first space 382A and second space 382B) may be filled with a thermally conductive adhesive, a thermally conductive epoxy, copper, steel, or any other thermally conductive material. In such a way, the thermally conductive material within each space (e.g., first space 382A and second space 382B) may contact and/or extend proximally through at least a portion of layer 384. For example, the thermally conductive material within first space 382A may contact first LED 374A and layer 384. Similarly, the thermally conductive material within second space 382B may contact second LED 374B and layer 384. In some examples, first space 382A and second space 382B may be filled with the same thermally conductive material. In other examples, first space 382A and second space 382B may be filled with different thermally conductive materials.

The presence of the one or more thermally conductive material(s) is illustrated schematically by clouds, for example, a first cloud 375A and a second cloud 375B in FIG. 4. The heat transferred from first LED 374A and second LED 374B is illustrated by arrows, for example, a first pair of arrows 379A and a second pair of arrows 379B. The heat generated by each LED (e.g., first LED 374A and/or second LED 374B) may be transferred from each LED, through the thermally conductive material of each space (e.g., first cloud 375A of first space 382A and/or second cloud 375B of second space 382B). The heat may then be absorbed or otherwise dissipated by layer 384 of insertion portion 330. As such, the temperature of each LED (e.g., first LED 374A and/or second LED 374B) and/or the area around each LED (e.g., distal tip 250) may be reduced during use of the one or more LEDs.

Layer 384 may be configured to act as a heat sink, for example, to absorb and/or dissipate heat generated by each of first LED 374A and second LED 374B. In some examples, gaps 385 may be formed within layer 384 of insertion portion 330, for example, between the strands forming the mesh or lattice configuration of layer 384. Gaps 385 may be configured to dissipate heat from each LED (e.g., similar to fins of a heat sink). In other examples, layer 384 may not include any gaps 385.

Although not shown, one or more additional layers may be disposed around layer 384 such that layer 384 is enclosed by, or otherwise at least partially insulated within insertion portion 330. In some examples, a distal portion of layer 384 may extend into distal tip 350 such that distal tip encloses or partially insulates the distal portion of layer 384. In these aspects, the additional layer(s) may be comprised of one or more insulating materials (e.g., polyvinyl chloride, thermoplastic polyurethane, polyetheretherketone, perfluoroalkoxy, ethylene tetrafluoroethylene, thermoplastic elastomers, or any other insulating material commonly used in the art to insulate medical devices). The additional layer(s) may be configured to protect the subject, for example, as layer 384 increases in temperature. For example, the temperature of layer 384 may increase as the heat is conducted away from the LEDs (e.g., first LED 374A and/or second LED 374B).

Additionally or alternatively, distal tip 350 and/or insertion portion 330 may be comprised of one or more materials, for example to assist in protecting the subject from the heat generated by the LEDs and absorbed by layer 384. For example, distal tip 350 and/or insertion portion 330 may be comprised of one or more insulating materials (e.g., polyvinyl chloride, thermoplastic polyurethane, polyetheretherketone, perfluoroalkoxy, ethylene tetrafluoroethylene, thermoplastic elastomers, or any other insulating material commonly used in the art to insulate medical devices). Additionally or alternatively, distal tip 350 and/or insertion portion 330 may be comprised of one or more layers of insulating materials, for example, surrounding an external surface of distal tip 350 and/or insertion portion 330. In these aspects, distal tip 350 and/or insertion portion 330 may radially surround at least a portion of layer 384. Additionally, distal tip 350 and/or insertion portion 330 may help to protect one or more components of medical device 15 (e.g., imaging device 372).

FIG. 5 illustrates an alternative configuration of a portion 455 comprising distal tip 450 and a distal portion of insertion portion 430. Distal tip 450 may have any or all of the characteristics of any of the distal tips previously described, except as described below. For example, distal tip 450 may include an imaging device 472 and/or one or more LEDs 474. For example, distal tip 450 may include a first LED 474A and a second LED 474B. Imaging device 472 and/or LEDs 474 may each be disposed on a distal face 450D of distal tip 450. Distal face 450D may be perpendicular to longitudinal axis A. In some examples, imaging device 472 may be disposed on distal face 450D, between first LED 474A and second LED 474B. Although not shown, alternative configurations of imaging device 472, first LED 474A, and/or second LED 474B may be utilized with distal tip 450. For example, first LED 474A may be disposed between imaging device 472 and second LED 474B. Additionally or alternatively, imaging device 472, first LED 474A, and/or second LED 474B may be disposed on one or more side surfaces 450S of distal tip 450.

Although not shown, distal face 450D may include one or more openings, for example, one or more working channels and/or lumens in insertion portion 430. Additionally, imaging device 472, first LED 474A, and/or second LED 474B may be positioned on distal face 450D in various positions relative to the one or more working channels and/or lumens.

In some examples, one or more articulation joints 486 may be disposed within an internal lumen (not shown) of insertion portion 430. The one or more articulation joints 486 may be configured so as to permit articulation of distal tip 450 in one or more directions. Articulation joint(s) 486 may extend proximally within insertion portion 430, for example, from a distal end of distal tip 450. Articulation joint(s) 486 may be configured as a heat sink, for example, configured to absorb heat generated by each of first LED 474A and second LED 474B. In such a way, articulation joint(s) 486 may be comprised of one or more thermally conductive materials (e.g., copper, stainless steel, aluminum, etc.). In some examples, one or more gaps 487 may be formed between each of the articulation joint(s) 486. For example, each gap(s) 487 may be configured to dissipate heat from each LED (e.g., similar to fins of a heat sink). In some examples, gap(s) 487 may be on alternating sides of insertion portion 430. For example, a first gap may be on a first side of insertion portion 430, and a second gap may be on a second side of insertion portion 430, opposite the first side. In other examples, articulation joint(s) 486 may not include any gaps 487 (e.g., each joint of articulation joint 486 may contact, or abut, one another).

As illustrated in FIG. 5, a first space 482A may be defined between first LED 474A and articulation joint(s) 486. For example, first LED 474A and articulation joint(s) 486 may be separated via first space 482A such that neither first LED 474A nor articulation joint(s) 486 are contacting one another. Similarly, a second space 482B may be defined between second LED 474B and articulation joint(s) 486 such that neither second LED 474B nor articulation joint(s) 486 are contacting one another.

In some examples, each of first space 482A and/or second space 482B may be filled with a thermally conductive material, for example, to assist in absorbing and transferring heat from each LED (e.g., first LED 474A and second LED 474B) to articulation joint(s) 486. For example, each space (e.g., first space 482A and second space 482B) may be filled with a thermally conductive adhesive, a thermally conductive epoxy, copper, steel, or any other thermally conductive material. In such a way, the thermally conductive material within each space (e.g., first space 482A and second space 482B) may contact each LED and articulation joint(s) 486. For example, the thermally conductive material within first space 482A may contact first LED 474A and articulation joint(s) 486. Similarly, the thermally conductive material within second space 482B may contact second LED 474B and articulation joint(s) 486. In some examples, first space 482A and second space 482B may be filled with the same thermally conductive material. In other examples, first space 482A and second space 482B may each be filled with a different thermally conductive material.

The presence of the one or more thermally conductive materials is illustrated schematically by clouds, for example, a first cloud 475A and a second cloud 475B in FIG. 5. The heat transferred from first LED 474A and second LED 474B is illustrated by arrows, for example, a first pair of arrows 479A and a second pair of arrows 479B. The heat generated by each LED (e.g., first LED 474A and/or second LED 474B) may be transferred from each LED, through the thermally conductive material(s) of each space (e.g., first cloud 475A of first space 482A and/or second cloud 475B of second space 482B). The heat may then be absorbed or otherwise dissipated by articulation joint(s) 486. As such, the temperature of each LED (e.g., first LED 474A and/or second LED 474B) and/or the area around each LED (e.g., distal tip 450) may be reduced during use of the one or more LEDs.

Although not shown, one or more additional layers may be disposed around articulation joint(s) 486 such that articulation joint(s) 486 are contained by, enclosed by, or otherwise at least partially insulated within distal tip 450 and/or insertion portion 430. For example, the additional layer(s) may be comprised of one or more insulating materials (e.g., polyvinyl chloride, thermoplastic polyurethane, polyetheretherketone, perfluoroalkoxy, ethylene tetrafluoroethylene, thermoplastic elastomers, or any other insulating material commonly used in the art to insulate medical devices). The additional layer(s) may be configured to protect the subject, for example, as articulation joint(s) 486 increases in temperature. For example, the temperature of articulation joint(s) 486 may increase as the heat is conducted away from the LEDs (e.g., first LED 474A and/or second LED 474B).

As previously discussed, distal tip 450 and/or insertion portion 330 may be comprised of one or more materials, for example to assist in protecting the subject from the heat generated by the LEDs and absorbed by layer 484. For example, distal tip 450 and/or insertion portion 430 may be comprised of one or more insulating materials (e.g., polyvinyl chloride, thermoplastic polyurethane, polyetheretherketone, perfluoroalkoxy, ethylene tetrafluoroethylene, thermoplastic elastomers, ceramic(s), or any other insulating material commonly used in the art to insulate medical devices). Additionally or alternatively, distal tip 450 and/or insertion portion 430 may be comprised of one or more layers of insulating materials, for example, surrounding an external surface of distal tip 450 and/or insertion portion 430. In these aspects, distal tip 450 and insertion portion 430 may radially surround articulation joint(s) 486. Additionally, distal tip 450 and/or insertion portion 430 may help to protect one or more components of medical device 15 (e.g., imaging device 472).

It will be apparent to those skilled in the art that various modifications and variations can be made to the disclosed device without departing from the scope of the disclosure. For example, it will be apparent to those skilled in the art that various aspects of each configuration discussed herein may be used with other configurations. For example, in some configurations, a distal portion of a medical device may comprise combinations of ferrules, steering cables, articulation joints, and/or thermally conductive layers of an insertion portion. In such a way, one or more components may be configured to absorb and/or dissipate heat generated by LEDs and/or other electrical components. By way of example, a distal portion of an exemplary medical device (not shown) may include ferrules (e.g., ferrules 180A, 180B) and/or an articulation joint (e.g., articulation joint 486) each formed of a thermally conductive material and configured to absorb and/or dissipate heat from the LEDs. Other configurations of a medical device may be contemplated.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device, comprising:
a handle,
an insertion portion coupled to the handle, wherein the insertion portion is configured to be inserted into a lumen of a subject; and
a distal tip coupled to a distal end of the insertion portion, wherein the distal tip includes a light emitting diode (LED) and a heat sink, wherein the heat sink is configured to absorb heat generated by the LED.

2. The medical device of claim 1, wherein the heat sink is disposed proximally of the LED in the distal tip.

3. The medical device of claim 1, wherein the heat sink includes a ferrule and an articulation wire, wherein the heat sink is coupled to a distal end of the articulation wire, wherein each of the ferrule and the articulation wire are comprised of one or more thermally conductive materials.

4. The medical device of claim 1, wherein a space is defined between the LED and the heat sink, wherein the space includes at least one of a thermally conductive material, a thermally conductive epoxy, or a thermally conductive adhesive.

5. The medical device of claim 4, wherein the at least one of the thermally conductive material, the thermally conductive epoxy, or the thermally conductive adhesive of the space is configured to contact both the LED and the heat sink.

6. The medical device of claim 2,
wherein the LED is a first LED, wherein the heat sink is a first heat sink,
wherein the distal tip further includes a second LED and a second heat sink, and
wherein the second heat sink is disposed proximally of the second LED.

7. The medical device of claim 6, wherein a first space is defined between the first LED and the first heat sink, wherein a second space is defined between the second LED and the second heat sink.

8. The medical device of claim 7, wherein the first space includes at least one of a first thermally conductive material, a first thermally conductive adhesive, or a first thermally conductive epoxy, and wherein the second space includes at least one of a second thermally conductive material, a second thermally conductive epoxy, or a second thermally conductive adhesive.

9. The medical device of claim 8, wherein the at least one of the first thermally conductive material, first thermally conductive epoxy, or the first thermally conductive adhesive of the first space contact both the first LED and the first heat sink, and wherein the at least one of the second thermally conductive material, the second thermally conductive epoxy, or the second thermally conductive adhesive of the second space contacts both the second LED and the second heat sink.

10. The medical device of claim 1, wherein the heat sink includes an articulation joint, wherein the articulation joint is configured to articulate the distal tip.

11. The medical device of claim 10, wherein the articulation joint includes one or more gaps.

12. The medical device of claim 10, wherein the articulation joint is disposed in a distal portion of the insertion portion.

13. The medical device of claim 10, wherein a space is defined between the LED and the articulation joint, wherein the space includes at least one of a thermally conductive material, a thermally conductive adhesive, or a thermally conductive epoxy.

14. The medical device of claim 1, wherein the heat sink includes at least one layer of the insertion portion, wherein the at least one layer includes a thermally conductive mesh.

15. The medical device of claim 14, wherein a space is defined between the LED and the at least one layer of the insertion portion, wherein the space includes at least one of a thermally conductive material, a thermally conductive adhesive, or a thermally conductive epoxy.

16. A distal end portion of a medical device, the distal end portion comprising:
a first light emitting diode (LED);
a second LED; and
a heat sink, wherein the heat sink is disposed proximally of the first LED and the second LED, wherein the heat sink is configured to absorb heat from the first LED and the second LED.

17. The distal end portion of claim 16, wherein a first space is defined between the first LED and the heat sink, wherein a second space is defined between the second LED and the heat sink, and wherein each of the first space and the second space include a thermally conductive adhesive.

18. The distal end portion of claim 17, wherein the thermally conductive adhesive of the first space contacts both the first LED and the heat sink, and wherein the thermally conductive adhesive of the second space contacts both the second LED and the heat sink.

19. A distal end portion of a medical device, the distal end portion comprising:
a first light emitting diode (LED);
a second LED;
a first heat sink, wherein the first heat sink is disposed proximally of the first LED; and
a second heat sink, wherein the second heat sink is disposed proximally of the second LED,
wherein each of the first heat sink and the second heat sink are configured to absorb heat from the first LED and the second LED, respectively.

20. The distal end portion of claim 19, wherein a first space is defined between the first LED and the first heat sink, and a second space is defined between the second LED and the second heat sink, wherein each of the first space and the second space include at least one of a thermally conductive adhesive, a thermally conductive epoxy, or a thermally conductive material.
